(19) United States
(12) Patent Application Publication
(10) Pub. No.: US 2012/0083688 A1
(43) Pub. Date: Apr. 5, 2012

(54) HEAD INJURY DIAGNOSTICS

(75) Inventors: Eric A. Nauman, West Lafayette, IN (US); Evan Breedlove, Lafayette, IN (US); Meghan Robinson, Lafayette, IN (US); Katherine E. Morigaki, West Lafayette, IN (US); Anne Zakrajsek, Lafayette, OH (US); Larry Leverenz, West Lafayette, IN (US); Tom Talavage, West Lafayette, IN (US)

(73) Assignee: PURDUE RESEARCH FOUNDATION, West Lafayette, IN (US)

(21) Appl. No.: 13/252,183

(22) Filed: Oct. 3, 2011

Related U.S. Application Data
(60) Provisional application No. 61/388,958, filed on Oct. 1, 2010.

(51) Int. Cl. A61B 5/055

(52) U.S. Cl. 600/419; 600/410

(57) ABSTRACT

Systems and methods for detecting the effects of concussion are disclosed. In various embodiments, fMRI or other live analysis technique captures data regarding the subject's brain activity while he or she is performing a task that exercises one or more particular functional centers. In some embodiments, post-collision activity is compared to baseline data, and the diagnosis operates as a function of the comparison. in other embodiments, post-collision activity is analyzed, such as by partitioning the activity data into regions of interest and calculating activity for one particular region in comparison with certain other, perhaps adjacent, regions.

### REFERENCE TO RELATED APPLICATION

This application is a nonprovisional of, and claims the benefit of U.S. Provisional Application No. 61/388,958, filed Oct. 1, 2010, which is hereby incorporated by reference in its entirety as if fully set forth.

### FIELD

The present invention relates to detection of brain injuries. More specifically, the present invention relates to application of functional MRI technology to detect concussions.

### BACKGROUND

Over one million young males play high school football in the United States each year, of which approximately 76,000 per year are clinically diagnosed with a concussion. More significantly, it is estimated that a similar number of concussed players go undiagnosed. Failure to diagnose concussions is a concern for two reasons. First, players with neurological damage not removed from play are at a higher risk for additional concussions. Second, biomechanics research has suggested that injury may be accumulated, a finding supported by histological evaluation of deceased athletes. Players who are not removed from play could thus accumulate injury in the form of multiple sub-concussive insults.

The effects of concussion—defined herein as a closed-head injury to the brain induced by mechanical insult—are part of the broader public concern about brain health. Concussion faults (traumatic brain injury, or TBI), which represents a significant component of brain health in the United States, with as many as 3.8 million sports-related incidents every year and approximately 50,000 deaths and 235,000 hospitalizations from all causes. Previous TBI has been shown to be a significant risk factor for repeat concussions and other neurological conditions including early-onset Alzheimer's disease, chronic depression, epilepsy, and chronic traumatic encephalopathy. At least 17% of individuals who experience multiple concussions develop chronic traumatic encephalopathy, or CTE, with some scientists suggesting that the incidence rate is likely higher. Athletes participating in sports involving a significant probability of head collisions, such as American football, represent a group that is at particularly high risk for concussion and other forms of TBI.

Currently, on-site healthcare professionals evaluate athletes for presence of concussion by examination for symptoms such as loss of consciousness, amnesia, headaches, dizziness, and inability to respond correctly to specific, direct questions. Drawbacks to this process include observation that symptoms often manifest themselves several hours after trauma, that symptoms do not clearly indicate a specific neurological dysfunction to treat, and that damage may accumulate over time as a result of injuries that do not produce symptoms meeting clinical criteria for concussions.

While concussion is inherently a mechanically induced injury, efforts to determine the underlying biomechanical mechanisms have been inconclusive. Attempts to correlate injury to kinematic input variables such as peak acceleration or the Head Injury Criterion have proven inadequate in their ability to accurately predict the occurrence of concussion. Similarly, efforts to identify metabolic factors that predispose an individual to concussion have remained elusive.

### BRIEF DESCRIPTION OF THE DRAWINGS

FIG. 1 is a chart of three groups of subjects of Phase 1 of operation of a first embodiment of the disclosed system and method, organized according to clinically observed impairment and functionally observed impairment.

FIG. 2 is a comparison of fMRI data from the left middle and superior temporal gyri for selected COI−/FOI− and COI+/FOI+ subjects before, during, and after the season.

FIG. 3 is a comparison of fMRI data from the left middle and superior frontal gyri for selected COI−/FOI− and COI−/FOI+ subjects before, during, and after the season.

FIG. 4 is a graph of acceleration events per player by region of the head, grouped by clinically and functionally observed impairment status.

FIG. 5 is a graph of the change in frontal lobe signal change versus head collision events exceeding 14.4G in practices and games in the preceding week.

FIG. 6 is a comparison for two selected players of fMRI activation in DLPFC associated with 2-back v. 1-back contrast for an N-back task.

FIG. 7 is an exemplary computer system for implementing many embodiments of the present system and method.

FIG. 8 is a longitudinal comparison of fMRI data for a first subject through Phases 1 and 2, with data regarding 1-back v. 0-back and 2-back v. 0-back assessments.

FIG. 9 is a longitudinal comparison of fMRI data for a second subject through Phases 1 and 2, with data regarding 1-back v. 0-back and 2-back v. 0-back assessments.

FIG. 10 is a flowchart showing a diagnosis and treatment method according to one embodiment of the disclosed system.

FIG. 11 is a flowchart showing a diagnosis and treatment method according to a second embodiment of the disclosed system.

### DESCRIPTION

For the purpose of promoting an understanding of the principles of the present system and method, reference will now be made to the embodiment illustrated in the drawings and specific language will be used to describe the same. It will, nevertheless, be understood that no limitation of the scope of the teaching is thereby intended; any alterations and further modifications of the described or illustrated embodiments, and any further applications of the principles of this teaching as illustrated therein are contemplated as would normally occur to one skilled in the art to which the teaching relates.

Generally, one form of the present system and method detects changes in functional MRI results during N-back tasks to identify patients who have experienced a concussion. Other forms use fMRI differences between adjacent regions of interest (ROIs) in the brain during N-back tasks to identify patients who have experienced a concussion, while still others use alternative tasks that exercise certain cognitive centers of interest. Yet others analyze structure information (such as DTI and/or SWI) or spectroscopy to examine the ROIs, either in a single operation or over longitudinal, multi-session data gathering.

### Phase 1

In order to model onset and development of cognitive impairment associated with head trauma in high school football, we monitored head collision events (HIT) experienced throughout the course of a single season, including practices and games, by 21 members of a high school varsity football team. Based on the number and nature of collision events, we longitudinally evaluated 11 of these athletes (8 varsity starters, 3 reserves) for changes in neurocognitive function and neurophysiology. Linking these findings, we have identified a group of high school football players without clinically observable signs of concussion who exhibited neurocognitive and neurophysiologic impairments comparable to or exceeding those exhibited by teammates who were diagnosed as concussed.

Materials and Methods

Subjects: Twenty-four (24) male high school football players (ages 15-18 years, mean=17.0) were enrolled. Twenty-one (21) participated in each aspect of the study throughout the 2009 season (Table 1). Of the three players who did not complete the study, two quit participation in football prior to the end of the season, and the third suffered a season-ending knee injury during the first game and did not participate in team activities thereafter.

TABLE 1
Dates and Results of IMPACT tests and dates of fMRI
assessments for all players enrolled in study

| | | | |
|---|---|---|---|
| | ImPACT ™ | ImPACT ™ | |
| | Memory | Memory | |
| ImPACT ™ | Composite | Composite | fMRI Session |
| Dates (s) | (Verbal) | (Visual) | Date(s) |

Player 100¹

| | | | | |
|---|---|---|---|---|
| Pre-Season | 5 August^{a} | 85 | 93 | 1 August |
| In-Season | 29 August^{b} | 75†, ‡ | 57†, ‡ | 29 August |
| Post-Season | 29 November^{b} | 93 | 68†, ‡ | 29 November |

Player 101

| | | | | |
|---|---|---|---|---|
| Pre-Season | 5 August^{a} | 93 | 81 | 1 August |

Player 102

| | | | | |
|---|---|---|---|---|
| Pre-Season | 5 August^{a} | 93 | 59 | 2 August |
| | 19 September | 96 | 56† | 19 September |
| In-Season | 1 October^{a} | 97 | 75 | |
| | 7 October^{b} | 83†, ‡ | 79 | 7 October |
| Post-Season | 23 November^{b} | 91†, ‡ | 79 | 23 November |

Player 103

| | | | | |
|---|---|---|---|---|
| Pre-Season | 5 August^{a} | 98 | 70 | 2 August |
| In-Season | 6 September^{b} | 82†, ‡ | 76 | 6 September |
| | 7 October^{b} | 78†, ‡ | 61†, ‡ | 7 October^{A} |
| Post-Season | 29 November^{b} | 84†, ‡ | 84 | 29 November |

Player 104²

| | | | | |
|---|---|---|---|---|
| Pre-Season | 5 August^{a} | 77 | 86 | 2 August |

Player 105

| | | | | |
|---|---|---|---|---|
| Pre-Season | 5 August^{a} | 87 | 67 | 2 August |
| In-Season | 18 October^{b} | 99 | 78 | 18 October |
| Post-Season | 21 November^{b} | 95 | 72 | 21 November |

Player 106

| | | | | |
|---|---|---|---|---|
| Pre-Season | 5 August^{a} | 90 | 81 | 2 August |

Player 107

| | | | | |
|---|---|---|---|---|
| Pre-Season | 5 August^{a} | 94 | 75 | 2 August |
| In-Season | 20 September^{b} | 99 | 83 | 20 September |

Player 108

| | | | | |
|---|---|---|---|---|
| Pre-Season | 5 August^{a} | 63 | 80 | 2 August |

Player 109²

| | | | | |
|---|---|---|---|---|
| Pre-Season | 5 August^{a} | 80 | 59 | 2 August |

Player 110³

| | | | | |
|---|---|---|---|---|
| Pre-Season | — | — | — | — |

Player 111

| | | | | |
|---|---|---|---|---|
| Pre-Season | 5 August^{a} | 84 | 70 | 2 August |

Player 112

| | | | | |
|---|---|---|---|---|
| Pre-Season | 5 August^{a} | 92 | 78 | 2 August |
| In-Season | 6 September^{b} | 97 | 80 | 6 September |
| Post-Season | 5 January^{a, c} | 86 | 77 | 18 November |

Player 113^{1, 2}

| | | | | |
|---|---|---|---|---|
| Pre-Season | 14 August^{a} | 67 | 85 | 2 August |

Player 114

| | | | | |
|---|---|---|---|---|
| Pre-Season | 7 August^{a, d} | 61 | 49 | 2 August |

Player 115

| | | | | |
|---|---|---|---|---|
| Pre-Season | 5 August^{a} | 94 | 73 | 3 August |
| In-Season | 5 September^{b} | 94 | 66† | 5 September |
| Post-Season | 18 November^{b} | 100 | 65† | 18 November |

Player 116

| | | | | |
|---|---|---|---|---|
| Pre-Season | 6 August^{a} | 98 | 95 | 3 August |

Player 117

| | | | | |
|---|---|---|---|---|
| Pre-Season | 5 August^{a} | 84 | 66 | 4 August |

Player 118¹

| | | | | |
|---|---|---|---|---|
| Pre-Season | 5 August^{a} | 91 | 75 | 4 August |
| In-Season | 18 October^{b} | 88†, ‡ | 61† | 18 October |
| Post-Season | 23 November^{b} | 96 | 84 | 23 November |

Player 119

| | | | | |
|---|---|---|---|---|
| Pre-Season | 5 August^{a} | 100 | 78 | 5 August |

Player 120

| | | | | |
|---|---|---|---|---|
| Pre-Season | 5 August^{a} | 88 | 96 | 5 August |
| | 29 August^{b} | 98 | 76†, ‡ | 29 August |
| In-Season | 10 October^{b} | 100 | 73†, ‡ | 10 October |
| Post-Season | 19 November^{b} | 93 | 75†, ‡ | 19 November |

Player 121

| | | | | |
|---|---|---|---|---|
| Pre-Season | 5 August^{a} | 77 | 91 | 6 August |
| | 26 September^{b} | 76 | 79† | 26 September |
| In-Season | 25 October^{b} | 88 | 70†, ‡ | 25 October |
| Post-Season | 23 November^{b} | 93 | 75†, ‡ | 23 November |

Player 122

| | | | | |
|---|---|---|---|---|
| Pre-Season | 6 August^{a} | 78 | 52 | 7 August |
| In-Season | 16 September^{b} | 91 | 68 | 16 September |
| Post-Season | 23 January^{b} | 89 | 81 | 23 January |

Player 123

| | | | | |
|---|---|---|---|---|
| Pre-Season | 11 August^{a} | 93 | 59 | 10 August |

Player Footnotes
¹Injured during season, did not return to play
²Quit participation in football prior to or during season
³Injured prior to practice, IMPACT not administered; HIT system monitored after return to play
ImPACT ™ Assessment Footnotes
^{a}Test administered at high school
^{b}Test administered at Purdue MRI Facility
^{c}Scores for test administered on day of fMRI Session not saved due to known on-line bug
^{d}Test flagged by ImPACT as possibly invalid
ImPACT ™ Score Footnotes
†Score outside 99% confidence interval
‡Score outside 99% confidence interval and flagged by IMPACT as significantly decreased
fMRI Assessment Footnotes
^{A}Computer network failure precluded completion of fMRI assessment; not included in analyses

Head Collision Event Monitoring: Participants in this study had Head Impact Telemetry (HIT) sensors from Simbex of new Lebanon, N.H., installed in their helmets. This system utilizes six accelerometers that provide three components each of linear and angular acceleration, measuring direction and intensity of collision events experienced by the head. Each set of sensors is equipped with a wireless transmitter that provides real-time telemetry to a nearby laptop, which records the linear accelerations and impact location for each event.

Pre-Season Assessment: Prior to the beginning of contact drills, 23 of the enrollees completed both pre-season neurocognitive (IMPACT) and neurophysiologic (fMRI) assessment to quantify individual and group baselines. Neurocognitive testing was conducted at the high school, either in groups of up to 10 players in the library (19/23) or individually at the desk of the athletic trainer (4/23).

Neurocognitive Testing: Functional MRI was performed at the Purdue MRI Facility (West Lafayette, Ind.) on a Signa HDx sold by 3T General Electric (Waukesha, Wis.). This system is equipped with real-time monitoring, permitting excessive (greater than 0.5 mm) within-acquisition motion to be identified on-site and acquisitions repeated as necessary until subject compliance is achieved. All 30-minute imaging sessions used a 16-channel brain array (Nova Medical, Wilmington, Mass.). For registration, whole-brain high-resolution images (3D-FSPGR; 1 mm isotropic resolution) were acquired, including the cerebellum.

Three functional runs were conducted of a visual working memory (N-back) paradigm using gradient-echo echo-planar imaging with TR/TE=1500/26 ms; matrix=64×64; FOB equals 20 cm; 34 slices; 3.8 mm thickness; 117 volumes). In each run, subjects performed one block (15 presentations, three-second interval, five targets per block) each of 0-, 1- and 2-back tasks for single letters. Visual presentation was via fiber-optic goggles (NordicNeuroLab, Bergen, Norway). Subjects responded by dominant index finger, via fiber-optic button box (Current Designs, Philadelphia, Pa.). Presentations and responses were implemented using E-Prime (Psychology Software Tools, Sharpsburg, Pa.). The order of the task blocks in the three rounds was counterbalanced, both within each session and across assessments.

In-Season Assessment: During each of the 10 weeks in the season, 1-3 players were invited to undergo in-season assessment. Players were invited if (a) they were diagnosed by the team physician as having experienced a concussion, (b) they were not identified by the physician is being concussed, but their HIT system data indicated they had accrued unusually large numbers of collision events or at least one high-magnitude (i.e., >100G) acceleration during that week's practices and game(s), and (c) athletes who participated in both practices and games but did not experience either a large number of collision events or a high-magnitude acceleration. Participant compliance with these invitations was 75%, and 15 in-season assessments were initiated. All 15 IMPACT assessments were completed. Due to a network malfunction, only 14 fMRI sessions were performed in whole and included in our analysis.

In-Season assessments were conducted within 48 hours of the game or 72 hours of diagnosis of concussion. IMPACT testing was conducted at the MRI Facility with the player isolated in an office, and fMRI was conducted as above. The 11 players undergoing in-season assessments included eight (8) who were invited on the basis of criteria (b) or (c) above. Players invited under criterion (b) were primarily recruited from among those who had accrued large numbers (i.e., top 25%) of head collision events, as assessed by the HIT. The three remaining players undergoing an in-season assessment represented three of the four players who were diagnosed by the team physician as having experienced a concussion. Note that one of the eight players receiving an in-season assessment while exhibiting no symptoms associated with concussion later received a diagnosed concussion, but declined to participate in further assessments. Because this player had not yet experienced a concussion at the time of the pre- and in-season assessment, his data have been included with the group of players who exhibited no symptoms of concussion.

Post-Season Assessment: Ten of the eleven players (excluding the player noted just above) who underwent in-season assessment returned 1-3 months after the end of the season for “post-season” assessment. IMPACT testing was conducted at the MRI Facility (for nine players) or in the high school athletic training room (one player). fMRI was conducted as above.

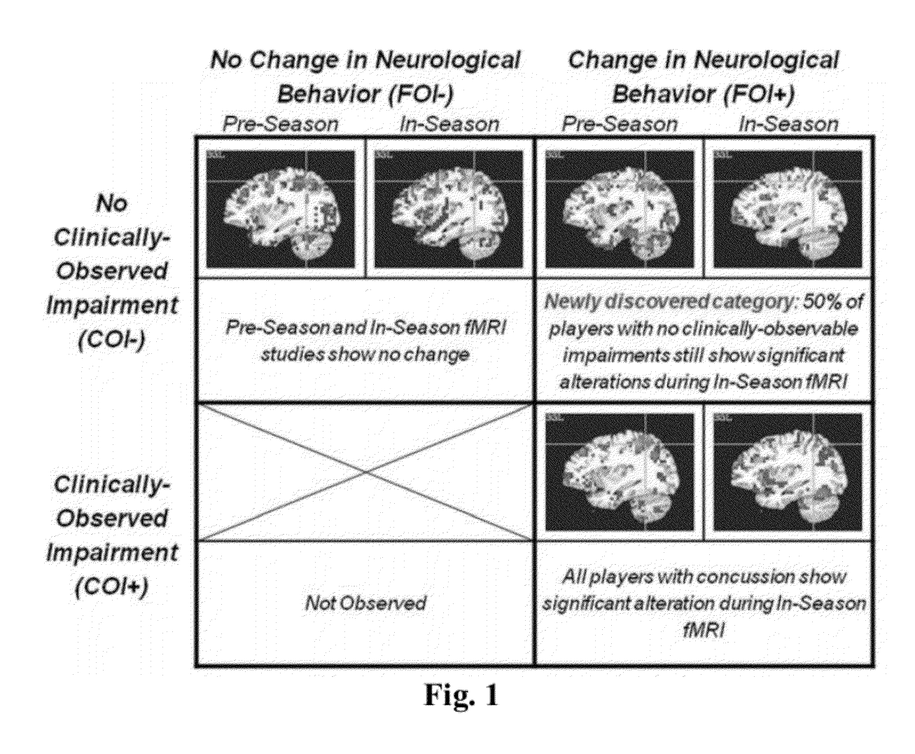

Player Categorization: Observed changes in neurologic health were subsequently examined in the context of clinical history of diagnosis or non-diagnosis of concussion during the course of the season and by detection or non-detection of abnormal re-test behavior using IMPACT. To this end, a 2×2 categorization matrix was defined for group evaluation (see FIG. 1). Players who were diagnosed by the team physician with a concussion were deemed to be positive for clinically observed impairment, and are labeled COI+. Players who were not diagnosed with a concussion were negative for this feature, and are labeled COI−. Players who exhibited deviant IMPACT re-tests were said to be positive for a functionally observed impairment (FOI+) IMPACT scores, while those whose IMPACT scores fell within the 99% confidence intervals were negative for this feature (FOI−).

Statistical Analysis: Three categories of data were evaluated in this work: neurocognitive scores (IMPACT), collision events (HIT system), and neurophysiologic signal changes (fMRI). In addition, the statistical significance of the observed frequencies of player categories was evaluated.

The consequences of the multiple environments in which IMPACT testing was performed were modeled via regression to most accurately identify abnormal re-test performance—the documented range of reliable test/re-test performance is based on a single site. Verbal and Visual Memory Composite scores from re-tests from which IMPACT did not indicate performance outside the reliability range were regressed on a population basis to compute the effect of site, permitting prediction of re-test performance at either site. Population variances were computed for IMPACT scores based on the pre-season tests conducted at the high school. 99% confidence intervals were generated around each player's re-test scores—on a site-specific basis—using the pre-season test variances scaled to account for observation of a higher mean for MRI Facility re-tests. Verbal and Visual Memory Composite re-test scores outside of these intervals were deemed to be “abnormal” (see Table 1). Note this approach is conservative, being biased toward non-detection of abnormal re-test performance.

Collision events recorded by the HIT system for each player were analyzed using a one-way ANOVA to identify differences between categories of players (see above). Observed differences were assessed for significance using a Bonferroni-corrected one-tailed t-test, with the alternative hypothesis being that COI−/FOI+ group exhibited the highest number of events under given location and magnitude constraints.

The fMRI data were analyzed using AFNI. Pre-processing included slice timing correction, motion correction, normalization to Talairach space, and 8 mm Gaussian smoothing for inter-subject comparison. Individual runs (no more than one per subject) were discarded if extensive mid-sagittal ventricular “activity” was observed, suggestive of stress-induced, stimulus-correlated changes in physiologic behavior (e.g., cardiac rate, respiratory cycle), likely arising from participants being uncomfortable in the MRI environment. Final analysis for each subject was effected on concatenated data, using a general linear model approach with Gamma Variate hemodynamic response function (without derivatives). The contrast of interest is a comparison between 2-back and 1-back working memory tasks, with statistically significant activation identified using a threshold of p<0.05, corrected for false discovery rate (FDR). Changes in fMRI activation were assessed using the 116 anatomically defined regions of interest (ROIs) from MarsBaR. For each player category (see below), a given ROI was said to exhibit significantly altered neurophysiologic activity if the mean t-statistic fell outside the 99.9% confidence interval derived for that ROI using the pre-season data (23 players) for both (a) the group fixed-effects mean, and (b) a majority of players with the group.

Cross-modality analyses were performed to assess whether subsequently observed changes in fMRI assessment of physiology were correlated with head collision events. To evaluate possible short-term neurophysiologic effects of head collision events, alteration of hemodynamic response signal amplitudes observed during in-season fMRI relative to that observed within the same subject in the pre-season assessment (i.e., % SignalChange_{In-season}−% SignalChange_{Pre-Season}) was compared to the number of head collision events measured by the HIT system in the week prior to the in-season assessment. This assessment was performed both on an anatomical ROI basis (i.e., for all 116 anatomical ROIs) and on a more global basis, for an aggregated ROI encompassing almost the entirety of the frontal lobe, excluding only the precentral gyrus (i.e., motor cortex, expected to be equally active in all tasks).

To document that the designated player categories were statistically meaningful, the predictive power of player category with respect to fMRI activation was evaluated using a one-tailed version of Fisher's Exact Test. Fisher's Exact Test is a more conservative version of the chi-square that is appropriate for smaller study populations. A 3×2 implementation of the test was used, where the player categories are considered the treatments, and the observation is either a decrease or non-decrease in the in-season frontal lobe hemodynamic response signal amplitude, relative to that obtained from the pre-season assessment, evaluated on a per-subject basis. The alternative was that the COI−/FOI+ player category was significantly associated with increased probability of observation of decreases in the aggregate frontal lobe response amplitude. The null hypothesis is that observation of decrease in aggregate frontal lobe response amplitude is not associated with the COI−/FOI+ categorization. Note that categorization was made from IMPACT scores without knowledge of the aggregate frontal lobe signal change.

Results

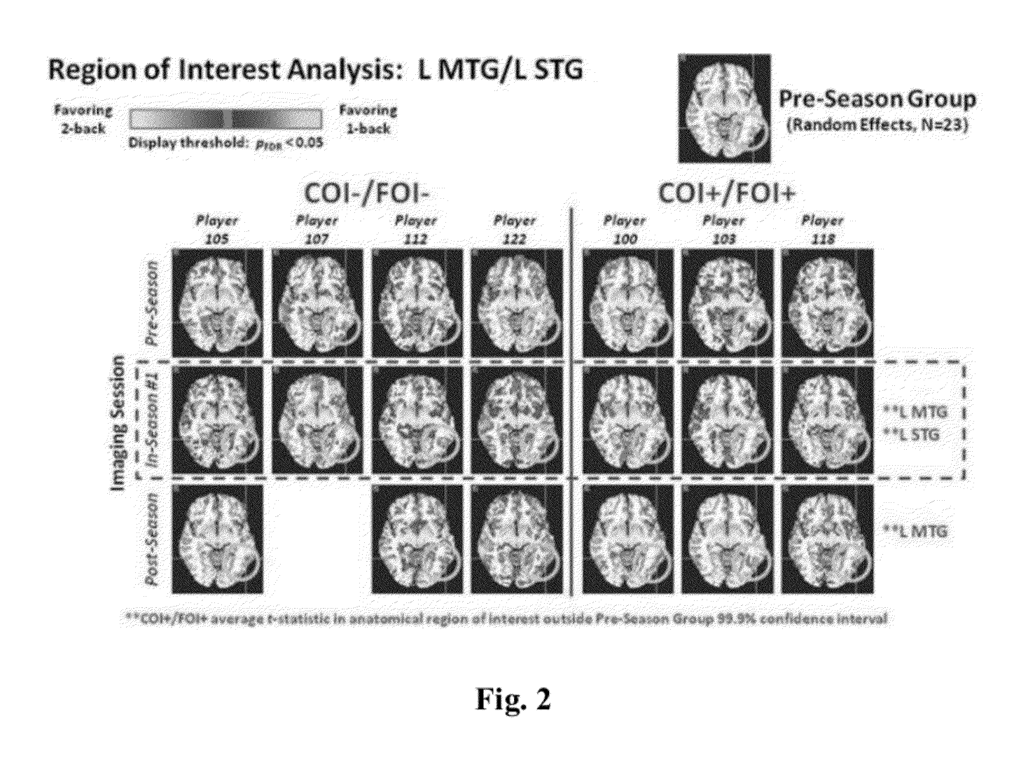

Four of the 21 full-season participants were diagnosed with a concussion (i.e., were COI+) as a consequence of activities related to a practice or a game. Three of these players participated in an in-season assessment within 72 hours of the diagnosis. One player (100) was obligated to cease participating in football due to persistent symptoms following the injury. A second player (118) was injured near the end of the season and was not cleared to play prior to the last game. A third player (103) missed one game and returned to play the following week. As expected, all three of these COI+ players examined within 72 hours of diagnosis of concussion were found to exhibit significantly lower neurocognitive performance in one or both of the Verbal and Visual Memory Composite scores on IMPACT. Based on joint observation of impairment by the team physician and the athlete's neurologic assessment scores, these players are categorized as COI+/FOI+. fMRI data for these players revealed alterations in the pattern and amplitude of signal differences observed when contrasting the 2-back and 1-back memory tasks, particularly in posterior middle and superior temporal gyri, regions associated with accessing linguistic representations of external stimuli (FIG. 2).

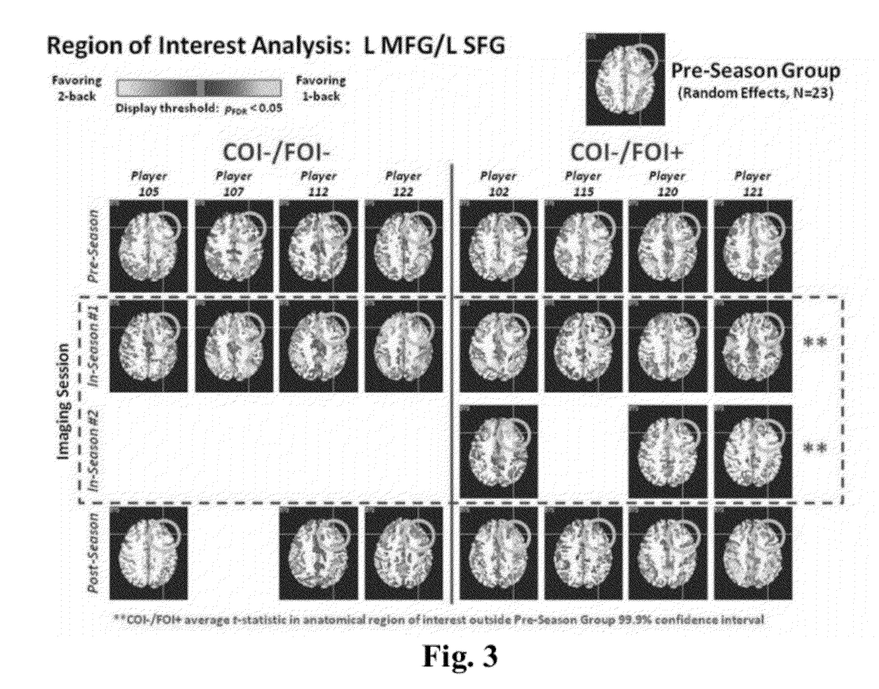

Four (105, 107, 112, 122) of the eight players invited to undergo in-season assessment in the absence of a clinical diagnosis of concussion (i.e., designated as COI−) exhibited no statistically significant deviations in IMPACT (see Table 1). These players were categorized as COI−/FOI−. The in-season fMRI data for this group remained consistent with pre-season evaluation in 115 of the 116 regions of interest, both on a within-player basis, and relative to the group random effects analysis (FIGS. 2-3). The only exception was in right cerebellum 3, which exhibited decreased activation in three players. Three of the four COI−/FOI− players completed participation in a post-season assessment, at which time IMPACT scores and task performance were again found to be within test/re-test limits.

Unexpectedly, four (102, 115, 120, 121) of the eight COI− players evaluated during the season, while exhibiting no symptoms that would prompt evaluation for concussion by the team healthcare personnel, were found to exhibit statistically significant reductions in IMPACT scores (Verbal and/or Visual Memory Composite scores; see Table 1). On this basis, these players are categorized as COI−/FOI+. This finding was augmented by observation (FIG. 3), in all such individuals at all in-season assessments (seven total, across four players), of significantly decreased fMRI activation levels in dorsolateral prefrontal cortex (DLPFC; middle and superior frontal gyri) and cerebellum, regions of the brain strongly associated with working memory. In particular, when the 2-back and 1-back working memory conditions were contrasted, activation in the DLPFC changed from favoring (i.e., being greater for) the 2-back condition, to favoring the 1-back condition (FIG. 2). Note that DLPFC has previously been documented to favor the 2-back condition in healthy controls and was also found to favor the 2-back condition in our participants who did not exhibit deviant IMPACT performance (i.e., COI−/FOI−; see FIGS. 2 and 3). When compared with those players clinically diagnosed as having been concussed (i.e., COI+/FOI+), the COI−/FOI+ players were found to be at least as impaired (demonstrated by both IMPACT and fMRI measures) as the known concussed group.

The observed player categories were found to be statistically meaningful with the null hypothesis rejected at the p<0.04 level (Fisher's Exact Test). Therefore, the COI−/FOI+ category is a justifiable segmentation of the subjects with respect to fMRI signal change.

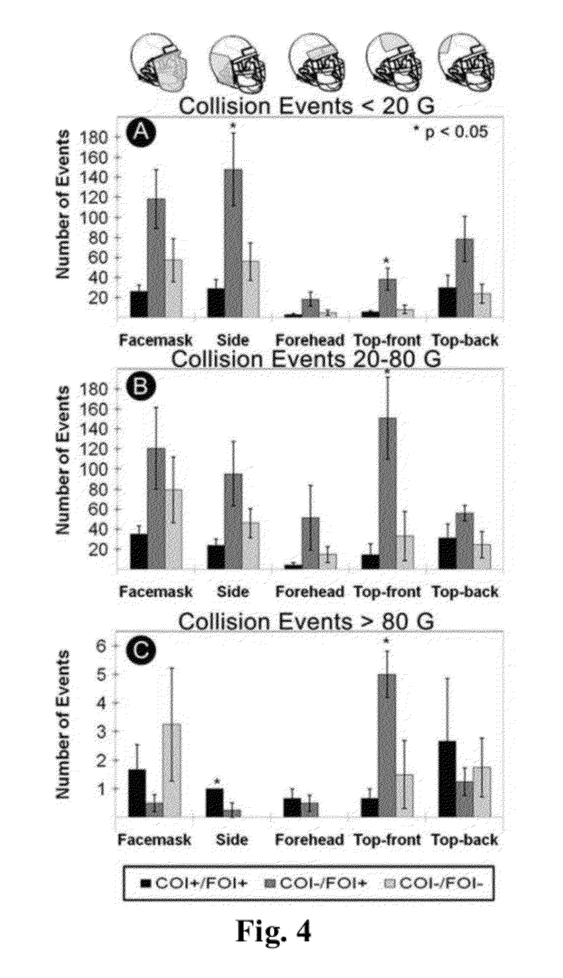

Evaluation of HIT system data indicated that the COI−/FOI+ group was different from the other two groups with regard to the total number and distribution of collision events. The 21 players participating in our study throughout the season experienced 15,264 collision events—i.e., a motion/action during which at least one accelerometer registered a magnitude in excess of 14.4G—across 48 practices and games (varsity and junior varsity; including pre-game warm-up sessions), an average of 15.5 collision events per player per organized activity. Among players who started for either the varsity or junior varsity, per player collision event totals ranged from a high of 1855 (Player 121; COI−/FOI+; 38.6 events per session) down to a low of 226 (Player 107; COI−/FOI−; 4.7 events per session). The total number of collision events experienced by the COI−/FOI+ group was significantly greater than any other group. The difference becomes even more pronounced when the number of collision events is examined on the basis of both region and magnitude (FIG. 4). Specifically, the COI−/FOI+ group exhibited more high-magnitude (greater than 80G) collision events directed to the top front of the helmet—immediately above the DLPFC in which functional changes were observed (FIG. 3).

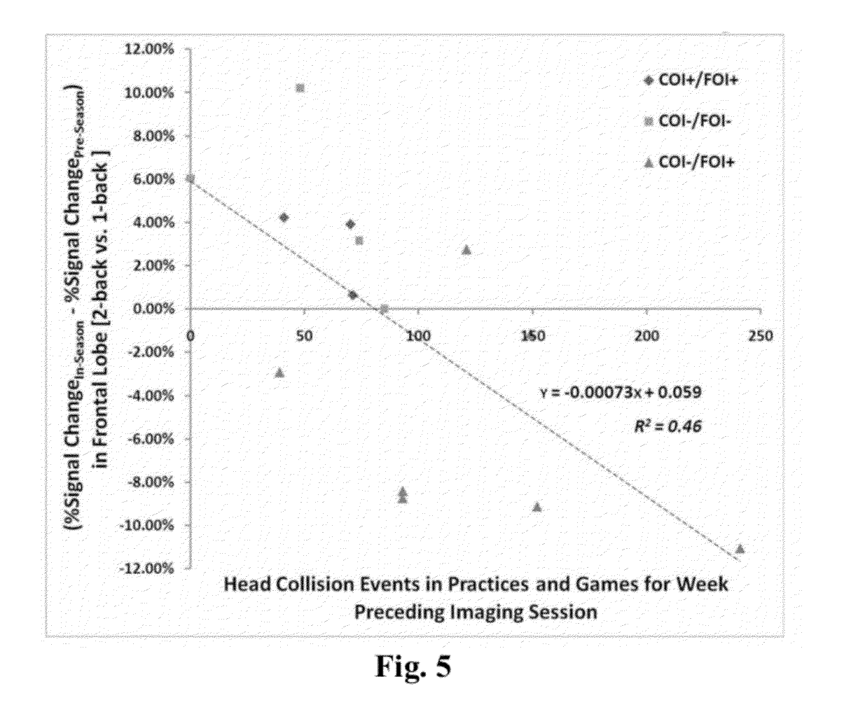

Further evaluation of the head collision events experienced by the 11 players assessed during the season revealed that the number of events experienced in the week immediately preceding and in-season assessment (N=14) was significantly correlated with changes in fMRI activation for the 2-back versus 1-back contrast of interest. At the level of anatomical ROIs (Table 2), statistically significant (p<0.05; |r|≧0.53) correlations were observed for collision events with the deviation of the 2-back versus 1-back signal change from that observed for the individual in the pre-season assessment. For all of the ROIs listed in Table 2, fMRI signal changes became less biased in favor of the 2-back task (i.e., lesser activation for the 2-back task, relative to that evidence for the 1-back task) as number of head collision events increased. While the most consistent changes in activation were located in the DLPFC, the majority of anatomical structures associated with this region (e.g., L MFG, L SFG, R SFG) are not indicated in Table 2. However, comparison of collision events to calculated changes in hemodynamic response signal amplitude for the aggregated frontal lobe ROI yields a trend that is well described by a linear regression model (R²=0.46; see FIG. 5). Entries annotated with (*) have p<0.01.

TABLE 2
Anatomical regions of interest from MarsBaR

| Anatomical Region of Interest | Correlation between fMRI |
|---|---|
| (MarsBaR ROI #) | Contrast and Collision Events |
| Frontal Medial Orbital L (41) | −0.70(*) |
| Frontal Medial Orbital R (42) | −0.72(*) |
| Frontal Middle R (46) | −0.55 |
| Frontal Superior Orbital L (50) | −0.71(*) |
| Fusiform R (54) | −0.56 |
| Hippocampus L (57) | −0.60 |
| Hippocampus R (58) | −0.68(*) |
| Parahippocampal R (76) | −0.58 |
| Rectus L (89) | −0.59 |
| Rectus R (90) | −0.62 |
| Temporal Superior Pole L (103) | −0.59 |
| Temporal Superior Pole R (104) | −0.53 |

Discussion

The disclosed system was used to evaluate neurocognitive and neurophysiologic deficits in high school football players as a function of head collision events, using pre-season baselines to quantify observed deficits. Athletes with collision event distribution similar to those diagnosed with concussion were originally intended to serve as controls—as opposed to non-athletes. Unexpectedly, half of these controls demonstrated both neurocognitive and neurophysiologic deficits, prompting the designation of a new group without observable signs of concussion who nevertheless exhibited cognitive impairments (COI−/FOI+; FIG. 1).

Athletes are a particularly high-risk population for TBI, especially amateur hockey and football players. Of the two, football is the more commonly played sport, with approximately 1.1 million high school participants in the United States during 2008-2009 (per http://www.nfhs.org). Each year, between 43,000 and 67,000 of these players are diagnosed with concussions. Unfortunately, many young athletes do not appreciate the seriousness of concussion and failed to self-report symptoms—sometimes intentionally, as they seek to remain on the field—likely doubling the number of actual concussions. Those with undiagnosed impairment to are not removed from play are of critical concern, because they continue to experience repeated head collision events.

In addition to players who do not report their symptoms, the results presented here indicate that additional athletes—those that would be considered COI−/FOI+—may be accruing damage that does not immediately result in symptoms that are typically observed by a clinician. The analysis performed using Fisher's Exact Test demonstrates that the COI−/FOI+ category is statistically distinct from the a priori expected COI+/FOI+ and COI−/FOI− categories. This analysis examines the probability of decreased aggregate frontal lobe activation, which is a region associated with working memory. The frontal lobe was a structure of interest because working memory scores from IMPACT were used to make the original categorizations. It is therefore notable that significant changes in signal amplitude and working memory structures were statistically more frequent in COI−/FOI+ subject. This correspondence is consistent with the hypothesis of the existence of the COI−/FOI+ category.

While our data do not provide statistical confidence that 50% of all COI− subjects will exhibit functional impairment—this proportion has inherent scatter that must be measured through several replicates across different player populations—we nevertheless observed that 4 of the original 23 volunteer subjects, about 17%, were COI−/FOI+, which is still a sufficiently large proportion to raise concern.

It is suspected that the COI−/FOI+ group comprises players who experienced neurologic trauma arising from repeated, sub-concussive head collision events, each of which likely produces sub-clinical stress on neural tissue. In this case, the players failed to accrue sufficient short-term damage to integrative neural systems that they exhibited externally observable symptoms. As such, these players continue to participate in practices and games throughout the season with neurocognitive and neurophysiologic impairments persisting over time (FIG. 3), but never exhibiting symptoms that would trigger evaluation by a healthcare professional. These players not only may be representative of the group associated with “unreported” concussions, but are also likely to have received repetitive, sub-concussive blows to the head, so they may have an increased likelihood of long-term neurodegeneration.

Of particular interest, this functionally (but not clinically) impaired group was primarily comprised of linemen, who experience helmet-to-helmet contact on nearly every play from scrimmage, often to the top front of the head. This finding of degraded neurological performance in the absence of classical symptoms of concussion is consistent with prior observation of CTE in the absence of a commensurate history of concussion in two ex-NFL offensive linemen and a defensive back.

Our observation of two groups (COI+/FOI+ and COI−/FOI+) exhibiting neurocognitive and neurophysiologic impairment that is distinguished by the presence or absence of externally observable behavioral symptoms implies that these groups have experienced injuries that differ by mechanism and associated location(s) of damage. The COI+/FOI+ group exhibit onset and extent of behavioral deficits consistent with damage to integrative centers of the brain associated with auditory (especially language) processing, with such damage likely produced in locations unique to each individual by a singular, deleterious collision event. In contrast, the COI−/FOI+ group predominantly exhibits behavioral deficits in working memory (predominantly visual), that likely are produced by repeated sub-clinical trauma to specific locations in the brain.

Regardless of the uncertainty surrounding the specific injury incurred in the COI−/FOI+, the similarities of the fMRI impairment associated with members of this group (FIG. 3) suggests that future work may be able to identify the underlying causes of deficits within this population. It is worth noting that previous studies involving positron emission tomography (PET) have observed that changes in metabolism associated with TBI are spatially diffuse relative to the actual site of mechanically induced injury, and not necessarily localized to regions experiencing (transient) ischemia. Therefore, alterations in fMRI signal changes may not take place at the precise location of mechanically induced injury, but these alterations would be an expected consequence of the changes in metabolism associated with damage. Thus, players experiencing clinically diagnosable concussions (i.e., COI+/FOI+) due to subject-specific injuries would not be expected to exhibit group-wise consistency in the alteration of fMRI activations, but players experiencing a specific injury (i.e., possibly the COI−/FOI+ group) could.

Initial assessment (FIG. 4) of the mechanical insults (as assessed by the HIT system) to the athletes in the two FOI+ groups indicates that they did, in fact, experience different collision event histories, and supports the above hypotheses regarding the potential for identifying a common underlying injury in the COI−/FOI+ group. Note that these data also support the argument that peak acceleration is not a sufficient measure to predict cognitive deficit. Currently, the location of the postulated injuries in the COI−/FOI+ group (DLPFC and other working memory brain areas) and their apparent focal behavioral effect make it difficult to identify this group on-site. If an individual has not suffered damage to integration centers associated with language, nor to auditory processing pathways, he is unlikely to exhibit the symptoms necessary for identification as being concussed. Further, if working memory deficits are sufficiently small, the individual may not be aware of the additional effort required to complete everyday tasks, perhaps only becoming aware that a deficit is present under the duress of probes such as neurocognitive tests.

The results of this Phase 1 study suggest that functional MRI is a valuable tool for detecting neurophysiologic deficit after head injury. To better evaluate the structure-function relationships that cause neurological damage, one may expand the range of neurological testing done with the MRI and add structural assessments such as diffusion tensor imaging and susceptibility-weighted imaging modalities.

This particular implementation of the disclosed system and method was strengthened by acquisition of baseline data prior to the commencement of athletic activities, greatly increasing the ability to detect changes at both an individual and group level. Despite the small sample size, a precise correlation was found between deficits observed using an established neurocognitive assessment tool (IMPACT) and neurophysiologic changes observed with fMRI during a verbal working memory task. Consistent with the hypothesis that the different observed cognitive and neurophysiologic deficits arise from distinct mechanical insult histories, significant differences were observed between groups of players categorized by changes in IMPACT score.

### Phase 2

In a second phase of this work, 30 members of a high school football team (ages 15-19) were monitored for blows to the head throughout the season using the HITS system. Sixteen of these players were also monitored for such blows during Phase 1, discussed above. Of the 30 enrollees, 20 underwent longitudinal assessment, being tested before and during the football season (28 total assessments, 4 due to clinically observed concussion); 2 participated in testing during the season, but not prior to it; 2 participated only in collision event monitoring; 6 participated only in pre-season testing.

All imaging in this phase was again performed on the 3T GE Signa HDx at the Purdue MRI Facility, using a 16-channel coil (from Nova Medical). Concern that the reported functional impairment may be caused by primary axonal damage motivated collection of DTI (25 angles; 32 4 mm-thick slices) and SWI (70 2 mm thick slices) data to evaluate structural health of the brains of participants. Players performed 0-, 1- and 2-back tasks (as discussed above and understood by those of skill in the relevant art) at near-ceiling levels throughout the season.

Processing: DTI and SWI data were evaluated by an expert. fMRI data were processed using AFNI. fMRI sessions for P107, P111, P207 and P209 were omitted either due to braces-related artifacts or having multiple ROIs with residuals greater than 2.5 standard deviations from the group mean.

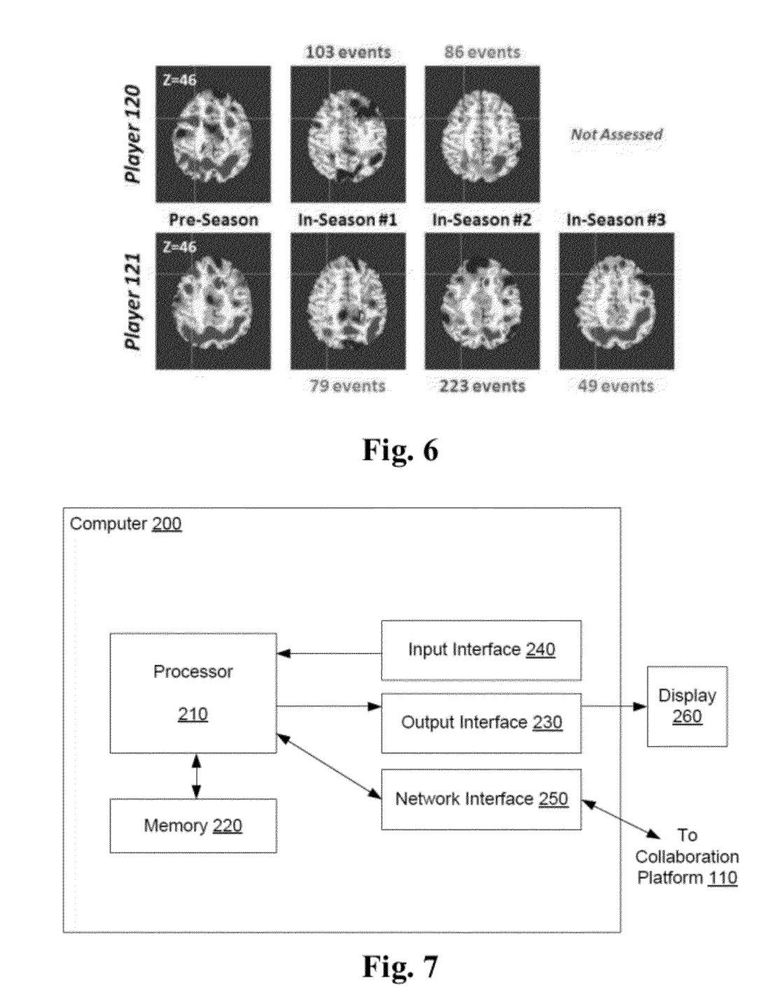

Results: As described above based on Phase 1, multiple regions of interest (ROIs) revealed statistically significant (p<0.05) anti-correlations between measured signal changes and number of blows to the head (range 0-223; mean 58.9) during the prior week: L and R MFG (both p<0.025), R SFG and R Inferior Operculum. FIG. 6 illustrates such signal changes at the level of the DLPFC for two linemen (P120, P121). No within-season changes were detected in DTI or SWI data for any players during Phase 2.

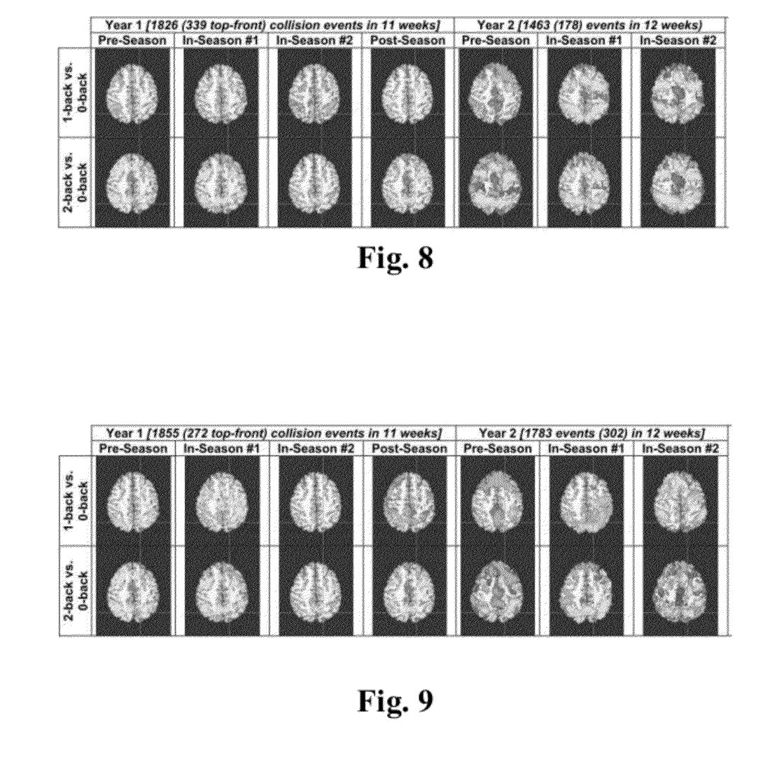

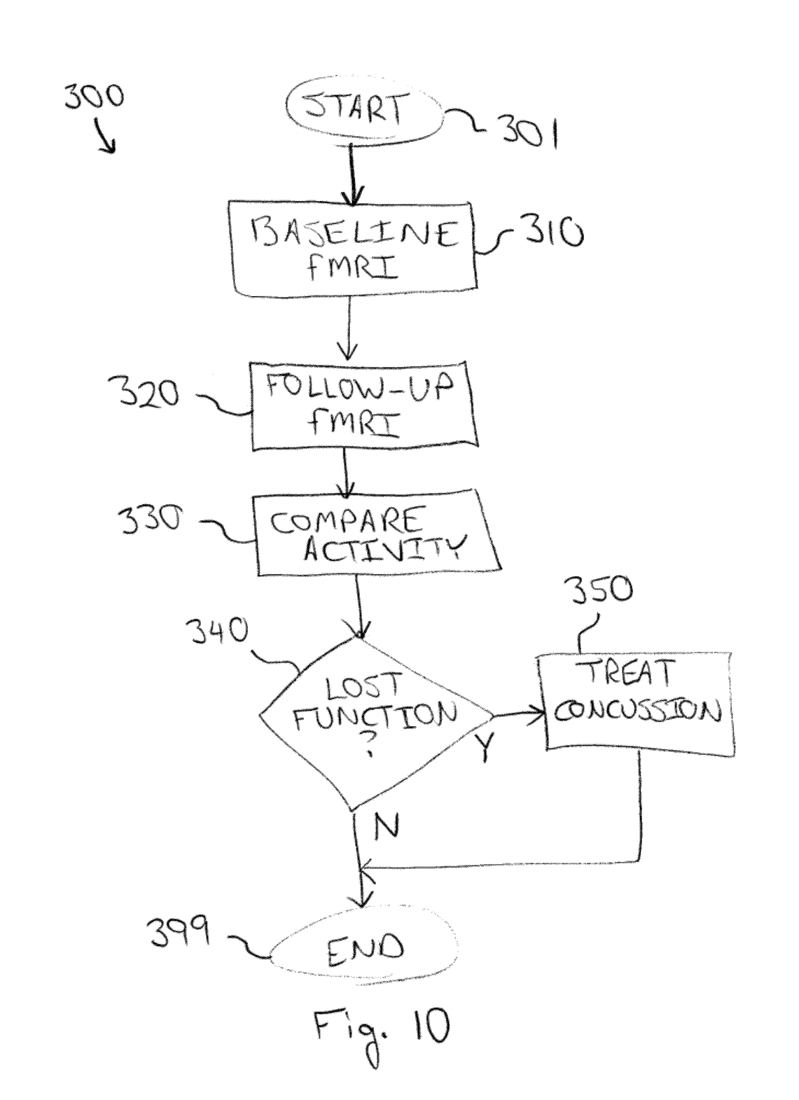

FIGS. 8 and 9 compare results for those lineman, P120 and P121, respectively, over the two phases of the study and in both 1-back v. 0-back and 2-back v. 0-back tasks. In Year 1, both players had accumulated a large number of blows to the head exceeding 14.4 g, with many of these blows on the top-front (P120: 1826 total, 339 top-front; P121: 1855 total, 272 top-front). While playing in one more game in Year 2, P121 experienced a comparable number of blows (1783 total, 302 top-front). P120 sought to improve his technique from Year 1, leading to decreased totals (1463 total, 178 top-front). In the week prior to in-season assessments, both players experienced more head blows in Year 1 than Year 2 (P120: 153 and 93 vs. 103 and 86; P121: 152 and 241 vs. 79 and 223). Neurocognitive Assessment: In Year 1, both players exhibited decreases in IMPACT Visual Composite (Memory) scores during in- and post-season assessments. In Year 2, P120 was found to exhibit no detectable change in any IMPACT score, whereas P121 again exhibited significant decreases at all in-season assessments.

Note that the depicted Year 2 data in FIGS. 8 and 9 have a higher signal-to-noise ratio, so direct comparisons are evaluated here only within a particular year. It was observed in Year 1 that changes in net fMRI activity in the frontal lobe were correlated with the number of blows experienced by the player during the week preceding assessment (R²=0.46). This trend was also observed for both players in Year 2 (FIGS. 8 and 9), with greater alteration in MFG/SFG observed for the in-season assessment following the greater accrual of collision events was experienced (P120: in-season #1; P121: in-season #2).

The observed changes in the recruitment of DLPFC in the chosen fMRI contrasts during in-season assessments associated with large numbers of head collisions suggest that these individuals are experiencing short-term impairment in their ability to restructure the visually presented letter stimuli to facilitate a more efficient processing strategy. Assessment of individual 1-back v. 0-back and 2-back v. 0-back maps (FIGS. 8 and 9) suggest that changes reported in the higher-level 2-back v. 1-back contrast arise due to increased activation (relative to pre-season) for the 1-back task coupled with reduced recruitment of networks involving the DLPFC for the 2-back task. The greater resemblance of P120's Year 2 (as opposed to Year 1) in-season data with the corresponding pre-season assessment, coupled with his non-decreasing IMPACT testing (i.e., no observed functional impairment) suggests that his technique alteration and the resulting reduction in head collisions has resulted in less trauma. Conversely, P121 continues to exhibit high variability suggesting continued accrual of trauma. These fMRI findings suggest that even if long-term damage is a consequence of reported functional impairment, the extent of such damage may be mitigated.

Phase 2 Conclusions: Current clinical practice results in non-participation by players who exhibit symptoms, but the newly observed functionally impaired group continues to play and receive blows to the head. Confirmation in Phase 2 of the findings from Phase 1 highlights the need to re-evaluate the practical definition of concussion and to raise awareness that the largest blows experienced by a player may not necessarily be those that produce the greatest long-term effect.

### Alternative Techniques

Various alternative embodiments use different means for identifying functional impairment of subjects. In some, fMRI data is gathered while the subject performs tasks other than N-back tasks that exercise certain functional centers of interest in the subject's brain. In others, structure information such as DTI and/or SWI is used to detect changes in function in the subject's brain. In still others, spectroscopy gives a picture of the chemical composition and activity of the brain, effectively providing data at a higher resolution than fMRI presently can.

Additional Information

Various embodiments are implemented on a computer of the form illustrated in FIG. 7. Computer 200, as this example will generically be referred to, includes processor 210 in communication with memory 220, output interface 230, input interface 240, and network interface 250. Power, ground, clock, and other signals and circuitry are omitted for clarity, but will be understood and easily implemented by those skilled in the art.

With continuing reference to FIG. 7, network interface 250 in this embodiment connects computer 200 a data network (such as a direct or indirect connection to Collaboration Platform 110) for communication of data between computer 200 and other devices attached to the network. Input interface 240 manages communication between processor 210 and one or more pushbuttons, UARTs, IR and/or RF receivers or transceivers, decoders, or other devices, as well as traditional keyboard and mouse devices. Output interface 230 provides a video signal to display 260, and may provide signals to one or more additional output devices such as LEDs, LCDs, or audio output devices, or a combination of these and other output devices and techniques as will occur to those skilled in the art.

Processor 210 in some embodiments is a microcontroller or general purpose microprocessor that reads its program from memory 220. Processor 210 may be comprised of one or more components configured as a single unit. Alternatively, when of a multi-component form, processor 210 may have one or more components located remotely relative to the others. One or more components of processor 210 may be of the electronic variety including digital circuitry, analog circuitry, or both. In one embodiment, processor 210 is of a conventional, integrated circuit microprocessor arrangement, such as one or more CORE 2 QUAD processors from INTEL Corporation of 2200 Mission College Boulevard, Santa Clara, Calif. 95052, USA, or ATHLON or PHENOM processors from Advanced Micro Devices, One AMD Place, Sunnyvale, Calif. 94088, USA, or POWER6 processors from IBM Corporation, 1 New Orchard Road, Armonk, N.Y. 10504, USA. In alternative embodiments, one or more application-specific integrated circuits (ASICs), reduced instruction-set computing (RISC) processors, general-purpose microprocessors, programmable logic arrays, or other devices may be used alone or in combination as will occur to those skilled in the art.

Likewise, memory 220 in various embodiments includes one or more types such as solid-state electronic memory, magnetic memory, or optical memory, just to name a few. By way of non-limiting example, memory 220 can include solid-state electronic Random Access Memory (RAM), Sequentially Accessible Memory (SAM) (such as the First-In, First-Out (FIFO) variety or the Last-In First-Out (LIFO) variety), Programmable Read-Only Memory (PROM), Electrically Programmable Read-Only Memory (EPROM), or Electrically Erasable Programmable Read-Only Memory (EEPROM); an optical disc memory (such as a recordable, rewritable, or read-only DVD or CD-ROM); a magnetically encoded hard drive, floppy disk, tape, or cartridge medium; or a plurality and/or combination of these memory types. Also, memory 220 is volatile, nonvolatile, or a hybrid combination of volatile and nonvolatile varieties.

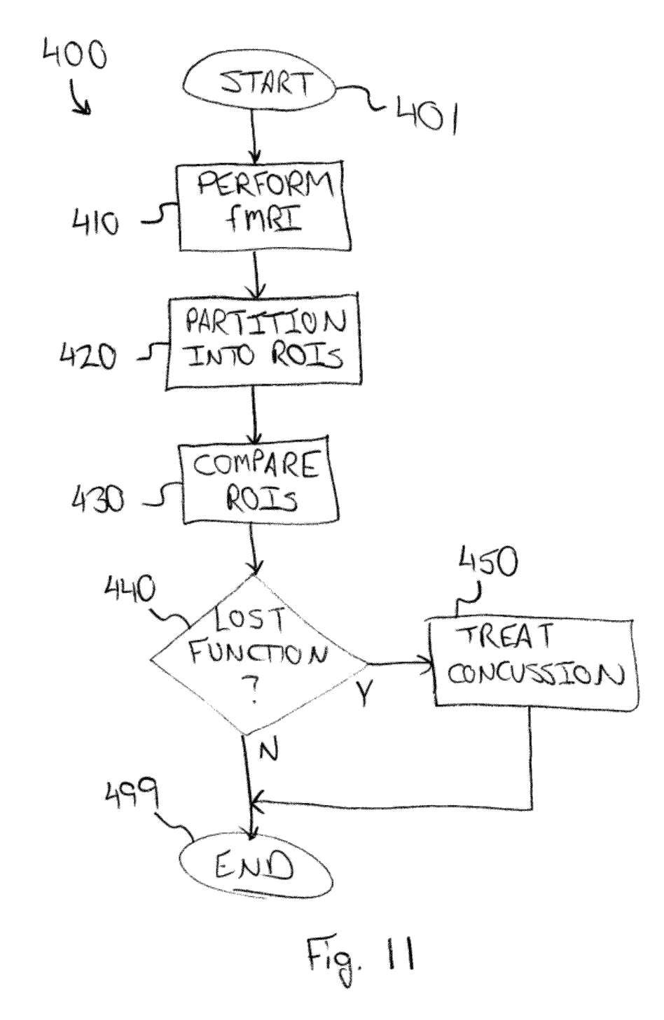

Thus, it might be seen that the general flow of some embodiments of the present systems and methods proceeds according to process 300, illustrated in FIG. 10. Process 300 starts (301) by performing a baseline fMRI (310) on the subject while he or she is engaged in a task that exercises one or more selected functional centers of his or her brain. The same subject later undergoes a follow-up fMRI (320) while engaged in the same or similar tasks. The output of the two fMRI sessions is then compared (330) according to the analysis described above. If the comparison indicates lost function (340), the subject is treated (350) for concussion. The process 300 ends (399).

Other embodiments, illustrated for example as process 400 in FIG. 11, avoid collection of baseline data. Process 400 begins (401) with performance of fMRI (410) or other activity-sensing scan on the subject while he or she is engaged in a task that exercises one or more selected functional centers of his or her brain. A processor (such as processor 210 illustrated in FIG. 7) partitions the fMRI (or other) data into regions of interest (420). It compares adjacent ROIs (430) and outputs a diagnostic conclusion (440). If the conclusion is that the subject has experienced a concussion, he or she is treated (450). The process 400 ends (499).

All publications, prior applications, and other documents cited herein are hereby incorporated by reference in their entirety as if each had been individually incorporated by reference and fully set forth. While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. A method of diagnosing a person for concussion, comprising:
taking a post-collision functional scan of a person's brain while the person is engaged in a task that exercises a particular part of the brain;
based on blood flow reflected in the post-collision functional scan, comparing activity in each of one or more particular regions of interest in the person's brain with activity in one or more spatially neighboring regions of the brain; and
diagnosing concussion based on the results of the comparison.

2. The method of claim 1, further comprising:
before taking the post-collision functional scan, taking a baseline functional scan of the person's brain while the person is engaged in the task; and
after taking the post-collision functional scan, comparing the baseline functional scan with the post-collision functional scan to identify changes in activity in one or more particular regions of interest, each as compared to changes in activity in one or more spatially neighboring regions of the brain.

3. The method of claim 2, wherein:
at least part of each functional scan is taken while the person is performing a task that stimulates a known functional processing center of the person's brain; and
the diagnosing step detects damage to the known functional processing center.

4. The method of claim 1, wherein the functional scans are functional MRIs.

5. The method of claim 1, wherein the functional scans are spectroscopic scans each operative to generate digital data indicative of chemical activity in the person's brain.

6. The method of claim 1, wherein the task is an N-back task.

7. A method of evaluating a person's brain health, comprising:
taking a baseline scan of a person's brain activity before a head trauma event;
taking a second scan of the person's brain activity after the event;
comparing the baseline scan with the second scan to identify changes in activity in one or more particular regions of interest, each as compared to changes in activity in one or more spatially neighboring regions of the person's brain; and
diagnosing a specific type of damage based on the results of the comparison.

8. The method of claim 7, wherein
the scans are functional MRIs taken while the person is engaged in a task that exercises a particular portion of the brain, and
the comparison is a function of changes in activity in the particular portion of the brain.

9. The method of claim 8, wherein the specific type of damage is damage to the particular portion of the brain.

10. The method of claim 8, wherein the task is an N-back task.

11. The method of claim 7, wherein the scans are spectroscopic scans each operative to generate digital data indicative of chemical activity in the particular portion of the brain.

12. A system for detecting concussion in a person, comprising: a processor and a memory in communication with the processor, the memory storing programming instructions executable by the processor to:
automatically compare activity represented by first functional MRI data from the person with activity represented by second functional MRI data from the person; and
display a diagnosis output as a function of the result of the comparison.

13. The system of claim 12, wherein
the programming instructions are further executable by the processor to partition data from the functional MRI of the person's brain into a plurality of regions of interest;
the first MRI data is taken from a first region of interest; and
the second MRI data is taken from a second region of interest.

14. The system of claim 12, wherein:
the first MRI data is from a baseline MRI acquired before a head trauma event; and
the second MRI data is from a different MRI acquired after the head trauma event.

15. The system of claim 12, wherein the first and second MRI data are each acquired while the person is engaged in a task that exercises a particular portion of the person's brain.

16. The system of claim 15, wherein the task is an N-back task.

17. The system of claim 15, wherein diagnosis output indicates whether the particular portion of the person's brain has experienced a concussion.
